(19) United States
(12) Patent Application Publication
(10) Pub. No.: US 2022/0273592 A1
(43) Pub. Date: Sep. 1, 2022

(54) ANALGESIC FORMULATION FOR PERINEAL PAIN

(71) Applicant: LANSINOH LABORATORIES, INC., ALEXANDRIA, VA (US)

(72) Inventors: Katherine Anne BOURDILLON, Leeds (GB); David Tom MCCAUSLAND, Leeds (GB); Rush Lloyd BARTLETT, II, Austin, TX (US); James Verlon CRONEY, Red Oak, TX (US)

(73) Assignee: LANSINOH LABORATORIES, INC., ALEXANDRIA, VA (US)

(21) Appl. No.: 17/650,555

(22) Filed: Feb. 10, 2022

Related U.S. Application Data
(60) Provisional application No. 63/147,954, filed on Feb. 10, 2021.

(51) Int. Cl. A61K 31/167; A61K 9/08; A61K 45/06

(52) U.S. Cl. CPC A61K 31/167; A61K 9/08; A61K 45/06

(57) ABSTRACT

An analgesic formulation for relief of postpartum perineal pain and discomfort is disclosed. The formulation generally includes an analgesic compound in a solution with herbal ingredients. The formulation has been optimized to provide a stable solution, free from precipitation. A method for treating a perineal area of a woman after giving birth can include: spraying a solution from a pump sprayer onto the perineal area, the solution comprising: a topical anesthetic; and at least one herbal ingredient.

### BACKGROUND

Birth causes a range of changes to the perineal area, some normal and some traumatic. These changes range from bruising, stretching and swelling, which are normal even after a straightforward birth, to episiotomies and tears, which may require stitching and are associated with pain and discomfort as they heal in the days and weeks following birth.

Lidocaine is a recognized topical treatment for the management of local muscular pain, and for reduction of pain and discomfort caused by hemorrhoids and other problems in the genital/anal area. Lidocaine is a local anesthetic that works by causing temporary numbness/loss of feeling in the skin and mucous membranes. Stability of lidocaine in aqueous solution is affected by a variety of factors, including pH, temperature and the presence of metal ions. When combined in solution with other ingredients, Lidocaine tends to form a precipitate, which affects stability of the resulting formulation.

Herbal solutions are also sometimes used to provide relief to injured perineal tissue, through astringent or anti-inflammatory properties. Some herbal ingredients, however, can irritate and/or sensitize the skin at higher concentrations or when applied incorrectly.

Therefore, there remains a need for an improved treatment for postpartum perineal pain and discomfort. Ideally, such a treatment would provide local analgesic pain relief, be quick acting, and free from side effects, such as damage to, or irritation of, the skin. At least some of these objectives will be addressed by this disclosure.

### SUMMARY

This disclosure describes a novel analgesic formulation for relief of postpartum perineal pain and discomfort. The formulation generally includes an analgesic compound in a solution with herbal ingredients. The formulation has been optimized to provide a stable solution, free from precipitation.

According to one aspect of the disclosure, the formulation contains lidocaine, at a concentration sufficient to give short term topical pain relief to the area of application. In some embodiments, the lidocaine is formulated along with herbal ingredients. These ingredients can include witch hazel, aloe vera, lavender, calendula extract, chamomile, tea tree oil, peppermint oil, sweet almond oil and/or any other suitable herbal ingredient(s). The formulation may also contain stabilizing ingredients to help stabilize the solution and keep it free from precipitation. The concentration of stabilizing ingredients within the formulation may increase the surface tension of the resulting solution to an optimized level, resulting in a drip-free coverage when sprayed on the skin, while still allowing the formulation to form a mist when aerosolized through a pump spray.

These and other aspects and embodiments are described in greater detail below.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

Local anesthetic sprays and creams are useful tools in managing peri-anal discomfort, itching and pain. Such treatments are also commonly repurposed for the perineal area to provide relief in the days and weeks after giving birth. Additionally, herbal ingredients have historically been used to soothe and relieve post-partum discomfort. Currently, however, no one device provides the benefits of topical anesthesia and herbal therapy together, in a stable formulation optimized to be safe for use in this sensitive area of the body.

The formulation described here contains a therapeutic concentration of lidocaine along with herbal extracts at concentrations characterized as safe for use on the sensitive perineal area, providing instant short term pain relief benefits alongside the known astringent, anti-inflammatory and healing benefits of the herbal ingredients. The formulation has an increased viscosity, due to the addition of stabilizing agents, which allows it to better adhere to the skin. Liquids that adhere well to the skin typically cannot form a mist when sprayed through a pump sprayer. A pump sprayer can include a container that holds the formulation, along with a pump-handle that pressurizes the container or otherwise allows the formulation to be sprayed from the container onto the desired area, in this case the perineal area.

The formulation described in this disclosure, however, can be designed to adhere to the skin while retaining the ability to mist when sprayed from a pump sprayer. Additionally, the formulation has been further optimized to be stable over a prolonged period of time (e.g., greater than 6 months under accelerated stability conditions or greater than 3 years under real time conditions) without lidocaine precipitating out of the solution.

The lidocaine concentration within the formulation is typically within the range of about 1-4% w/v. In alternative embodiments, the concentration of lidocaine is within the range of about 0.05%-5%. In other alternative embodiments, lidocaine is replaced with another topical local anesthetic at therapeutic concentrations. Examples of such anesthetics include, but are not limited to, Articaine, Benzocaine, Bupivacaine, Chloroprocaine, Cocaine, Dibucaine, Dyclonine, Etidocaine, Lignocaine, Mepivacaine, Prilocaine, Procaine, Tetracaine, and Xylocaine.

To form the solution described herein, lidocaine may be combined with one or more herbal ingredients, at individual concentrations of <5% v/v. These ingredients can include Hamamelis virginiana (witch hazel) Extract, Aloe Barbadensis Leaf juice, Lavender Oil, Calendula officinalis Flower Extract, Chamomile Recutita (Matricaria) Flower Extract, Tea Tree leaf oil, Mentha Piperita (peppermint) oil, Prunus amygdalus dulcis (sweet almond) oil and/or any other suitable herbal ingredient(s). Optionally, these herbal ingredients may be combined with or substituted by a solution containing one or more active antimicrobial agents, to prevent infection and/or manage microbial bioburden in the perineal area. Examples of such antimicrobial agents include, but are not limited to, iodine, Chlorhexidine, honey, PHMB, silver compounds (e.g., silver nitrate, silver sulphadiazine, or silver acetate), antibiotics such as but not limited to bacitracin and polymyxin, hyaluronic acid, and octenidine.

In some embodiments, the lidocaine and herbal ingredient(s) solution may also include one or more ingredients with stabilizing, emulsifying or preservative properties, at concentrations sufficient to result in a stable, precipitate free, clear solution. Examples of such additive ingredients include, but are not limited to, Caprylyl/Capryl Wheat Bran/Straw Glycosides, Fusel Wheat Bran/Straw Glycosides, Cellulose gum, Poloxamer1, PEG 40 Hydrogenated Castor Oil, Polyglyceryl-5 Oleate, Sodium Cocoyl Glutamate, Glyceryl Caprylate, Sodium phytate, EDTA, Sodium benzoate, Potassium Sorbate, Potassium benzoate, Poloxamer 188, Sorbic acid, Calcium sorbate, and calcium propionate. In some embodiments, the resulting solution is opaque but is still free or substantially free of precipitates. The resulting solution may also have increased viscosity. For example, the viscosity of the formulation may be within the range of about 0.0095-1.5000 poise at 25° C. In other embodiments, the viscosity of the formulation may be within the range of about 0.0095-3.0000 poise. In some embodiments, the resultant formulation is still able to be sprayed through a pump sprayer to give a fine and consistent mist, with a round spray pattern of approximately 8-9 cm from 15 cm distance, spray of liquid, or foam.

One specific embodiment of a proposed spray product may contain between about 2% w/v and about 4% w/v lidocaine and be co-formulated with the following herbals, including but not limited to: Hamamelis virginiana (witch hazel extract) up to 5.00%, Calendula officinalis flower extract (up to 0.50%), Chamomile Recutita (Matricaria) flower extract (up to 0.5%), Aloe Barbadensis leaf juice (up to 0.5%), Lavendula angustifolia (lavender) oil (up to 0.5%), Melaleuca alternifolia (tea tree) leaf oil (up to 0.50%), Mentha Piperita (peppermint) oil (Up to 0.50%), and or Prunus amygdalus dulcis (sweet almond) oil (up to 0.50%).

In some embodiments, the proposed formulation is adjusted to a pH of 4.0-5.0.

### Examples

Certain embodiments of the present disclosure are further described with reference to the following experiments and examples. These experiments, examples, and samples are intended to be merely illustrative of the disclosure and are not intended to limit or restrict the scope of the present disclosure in any way and should not be construed as providing conditions, parameters, reagents, or starting materials that must be utilized exclusively in order to practice the art of the present disclosure.

A clinical study on the present compositions and comparison against a commercial product Dermoplast® spray was conducted. A pain relieving spray composition (hereinafter “the tested sample”) according to Table 1 was prepared and tested in the clinical study. The dermal and gynecological irritation potential and consumer perception results were obtained and evaluated.

TABLE 1
Composition of the tested pain relieving spray sample in the clinical study.

| | | | Concentration |
|---|---|---|---|
| | | | (weight %) of |
| Ingredients | Chemical Name | CAS No. | ingredient |
| Deionized Water | Water (Aqua, Eau) | 7732-18-5 | 87.39400 |
| SDA Alcohol 40-B | Alcohol | 64-17-5 | 5.00000 |
| 200pf | | | |
| | T-Butyl Alcohol | 75-65-0 | |
| Lidocaine | Lidocaine Hydrochloride | 6108-05-0 | 4.00000 |
| Hydrochloride | | | |
| PEG-40 Hydrogenated | PEG-40 Hydrogenated | 61788-85-0 | 1.50000 |
| Castor Oil | Castor Oil | | |
| Witch Hazel 14% | Hamamelis Virginiana | 84696-19-5 | 1.00000 |
| Alcohol | (Witch Hazel) Extract | | |
| | Alcohol | 64-17-5 | |
| Pluracare ® F 68 NF | Poloxamer 188 | 9003-11-6 | 0.50000 |
| Potassium Sorbate | Potassium Sorbate | 7757-83-7 | 0.20000 |
| Sodium Benzoate | Sodium Benzoate | 532-32-1 | 0.20000 |
| Aquaion ™ 7H3SXF | Cellulose Gum | 9004-32-4 | 0.10000 |
| Cellulose Gum | | | |
| Dermofeel PA-12 | Sodium Phytate | 14306-25-3 | 0.05000 |
| Lavender Oil | Lavandula Angustifolia | 8000-28-0 | 0.03000 |
| | (Lavender) Oil | | |
| Peppermint Oil | Mentha piperita | 8006-90-4 | 0.01000 |
| | (Peppermint) Oil | | |
| Eugenol | Eugenol | 97-53-0 | 0.01000 |
| Aloe Vera Gel Spray | Aloe Barbadensis Leaf | 85507-69-3 | 0.00500 |
| Dried Powder 200X | Juice | | |
| Sweet Almond Oil | Prunus Amygdalus (Sweet | 8007-69-0 | 0.00100 |
| | Almond) Oil | | |
| Total | | | 100.00000 |

Study Design

Thirty-three female subjects were enrolled in this clinical study to evaluate the dermal and gynecological irritation potential and consumer perception of the tested sample. Study evaluations included dermal evaluations, gynecological evaluations, and consumer perception questionnaires.

Test Method

The test sample was sprayed onto the affected area of each subject from a sprayer 6 to 12 inches away from the affected area. The spray nozzle was directed towards skin to activate spray. Excessive amounts of the spray was avoided on broken skin, blistered, abraded skin, or open wounds. The tested sample was applied externally to the affected area not more than 3-4 times daily. Contact with eyes was avoided. The samples were tested on week 2 and week 5 respectively for each participant in the clinical study.

Subjects were blinded to the name of the test material. The investigatory staff was not blinded, with the exception of the clinical nurse, who was blinded at the time of grading. Test materials were labeled with unique CRL study identification and panel codes and subject numbers upon test material receipt by CRL.

Daily diaries were reviewed by clinic staff to confirm study compliance.

The external genitalia (labia minora, labia majora and vestibule/perineum) were each examined for signs of irritation including erythema, edema, and dryness by a clinical nurse. Any observed irritation were be graded and the results recorded on the dermal examination score sheet using a scoring scale.

Test Results

A total of 31 subjects completed the study. Two subjects (#05 and #33) were lost to follow up. Tables 2 and 3 show questionnaire results for the tested sample and Dermoplast® spray respectively.

TABLE 2
Questionnaire results for the tested pain relieving spray sample according to Table 1.

| | Most | Least | | |
|---|---|---|---|---|
| | Favorable | Favorable | | |
| Question | Percentage* | Percentage* | Z-score | Significant |
| Q1 Please rate how this product felt | 74.2% | 25.8% | 2.69 | Yes |
| when you first applied it to your skin, on | | | | |
| a scale from 0 to 10 (where 0 is very | | | | |
| unpleasant and 10 is very pleasant) | | | | |
| Q3 How well did the level of numbing | 96.8% | 3.2% | 5.21 | Yes |
| meet your expectations for a post-birth | | | | |
| product? | | | | |
| Q4 How much do you agree or disagree | 96.8% | 3.2% | 5.21 | Yes |
| with the following statement? ‘The | | | | |
| product felt exactly how I would want a | | | | |
| post-birth intimate product to feel when | | | | |
| applied to the skin’ | | | | |
| Q5 How would you rate the ease of | 93.5% | 6.5% | 4.85 | Yes |
| application for this product? | | | | |
| Q6 How would you rate the | 96.8% | 3.2% | 5.21 | Yes |
| performance of the nozzle/spray? | | | | |
| Q7 Please rate the scent of the product. | 77.4% | 22.6% | 3.05 | Yes |
| (where 0 is very unpleasant and 10 is | | | | |
| very pleasant) | | | | |
| Q9 How much do you agree or disagree | 90.3% | 9.7% | 4.49 | Yes |
| with the following statement?' The | | | | |
| product smelled exactly how I would | | | | |
| want a post-birth intimate product to | | | | |
| smell' | | | | |
| Q10 Thinking back to the birth of your | 93.5% | 6.5% | 4.85 | Yes |
| youngest child, do you think this | | | | |
| product would have offered relief in the | | | | |
| immediate post birth period? | | | | |
| Q11 Would you recommend this | 93.5% | 6.5% | 4.85 | Yes |
| product to friends or family? | | | | |
| Q14 if you were to give birth again, how | 93.5% | 6.5% | 4.85 | Yes |
| likely would you be to use this product? | | | | |

*Most Favorable Percentage = % of subjects chose 6-10 for Q1, Q7, % of subjects chose 1-3 for Q3, % of subjects chose 1-3 plus half of % of subjects chose 4 for Q4, Q5, Q6, Q9, % of subjects chose 1, 2, and half of 3 for Q10, Q11 and % of subjects chose 1, 2 for Q12;
** Least Favorable Percentage = % of subjects chose 1-5 for Q1, Q7, % of subjects chose 4-6 for Q3, % of subjects chose 5-7 plus half of % of subjects chose 4 for Q4, Q5, Q6, Q9, % of subjects chose 4, 5, and half of 3 for Q10, Q11 and % of subjects chose 3, 4 for Q12.

TABLE 3
Questionnaire results for Dermoplast ® spray.

| | Most | Least | | |
|---|---|---|---|---|
| | Favorable | Favorable | | |
| Question | Percentage* | Percentage* | Z-score | Significant |
| Q1 Please rate how this product felt | 77.4% | 22.6% | 3.05 | Yes |
| when you first applied it to your skin, on | | | | |
| a scale from 0 to 10 (where 0 is very | | | | |
| unpleasant and 10 is very pleasant) | | | | |
| Q3 How well did the level of numbing | 90.63% | 9.7% | 4.49 | Yes |
| meet your expectations for a post-birth | | | | |
| product? | | | | |
| Q4 How much do you agree or disagree | 91.9% | 8.1% | 4.67 | Yes |
| with the following statement? 'The | | | | |
| product felt exactly how I would want a | | | | |
| post-birth intimate product to feel when | | | | |
| applied to the skin' | | | | |
| Q5 How would you rate the ease of | 100.0% | 0 | 5.57 | Yes |
| application for this product? | | | | |
| Q6 How would you rate the | 100.0% | 0 | 5.57 | Yes |
| performance of the nozzle/spray? | | | | |
| Q7 Please rate the scent of the product. | 74.2% | 25.8% | 2.69 | Yes |
| (where 0 is very unpleasant and 10 is | | | | |
| very pleasant) | | | | |
| Q9 How much do you agree or disagree | 80.6% | 19.4% | 3.41 | Yes |
| with the following statement?' The | | | | |
| product smelled exactly how I would | | | | |
| want a post-birth intimate product to | | | | |
| smell' | | | | |
| Q10 Thinking back to the birth of your | 93.5% | 6.5% | 4.85 | Yes |
| youngest child, do you think this | | | | |
| product would have offered relief in the | | | | |
| immediate post birth period? | | | | |
| Q11 Would you recommend this | 95.2% | 4.8% | 5.03 | Yes |
| product to friends or family? | | | | |
| Q14 if you were to give birth again, how | 96.8% | 3.2% | 5.21 | Yes |
| likely would you be to use this product? | | | | |

*Most Favorable Percentage = % of subjects chose 6-10 for Q1, Q7, % of subjects chose 1-3 for Q3, % of subjects chose 1-3 plus half of % of subjects chose 4 for Q4, Q5, Q6, Q9, % of subjects chose 1, 2, and half of 3 for Q10, Q11 and % of subjects chose 1, 2 for Q12;
** Least Favorable Percentage = % of subjects chose 1-5 for Q1, Q7, % of subjects chose 4-6 for Q3, % of subjects chose 5-7 plus half of % of subjects chose 4 for Q4, Q5, Q6, Q9, % of subjects chose 4, 5, and half of 3 for Q10, Q11 and % of subjects chose 3, 4 for Q12.

Under the conditions of the present study and in this test population, the tested sample did demonstrate a positive subject opinion and elicited a statistically favorable consumer perception response in a majority of subjects. The questionnaire results provided Table 2 indicate that the preference for the tested sample by a majority of participants. Table 4 provides more consumer perception for the tested sample and the Dermoplast® spray product. Table 5 provides results of the binomial test for the preference questions comparing the test sample against Dermoplast® spray. A significantly higher percentage of subjects preferred the tested sample than the Dermoplast® spray for scent.

TABLE 4
Questionnaire results for the tested sample compared with Dermoplast ® spray.

| | The tested sample | | Dermoplast ® spray | |
|---|---|---|---|---|
| Question | Number | Percentage | Number | Percentage |

Q2. What word(s) best describe the way the product felt when you applied it to your skin?

| | | | | |
|---|---|---|---|---|
| Cooling | 18 | 58.1% | 18 | 58.1% |
| Soothing | 18 | 58.1% | 11 | 35.5% |
| Sticky | 3 | 9.7% | 2 | 6.5% |
| Smooth | 14 | 45.2% | 9 | 29% |
| Too cold | 2 | 6.5% | 2 | 6.5% |
| Clean | 14 | 45.2% | 12 | 38.7% |
| Thick | 1 | 3.2% | 1 | 3.2% |
| Thin | 10 | 32.3% | 4 | 12.9% |
| Greasy | 0 | 0 | 1 | 3.2% |
| Oily | 1 | 3.2% | 4 | 12.9% |
| Drying | 0 | 0 | 2 | 6.5% |

Q8 Which of the following words would you use to describe the scent? Please select all that
apply

| | | | | |
|---|---|---|---|---|
| Clean | 15 | 48.4% | 18 | 58.1% |
| Perfumed | 4 | 12.9% | 9 | 9 |
| Fresh | 15 | 48.4% | 12 | 38.7% |
| Pungent | 1 | 3.2% | 0 | 0 |
| Light | 10 | 32.3% | 8 | 25.8% |
| Sweet | 0 | 0 | 1 | 3.2% |
| Moist | 4 | 12.9% | 3 | 9.7% |
| Floral | 1 | 3.2% | 1 | 3.2% |
| Feminine | 8 | 25.8% | 6 | 19.4% |
| Earthy | 4 | 12.9% | 3 | 9.7% |
| Faint | 2 | 6.5% | 6 | 19.4% |
| Other | 1 | 3.2% | 1 | 3.2% |

TABLE 5
Comparative results of the binomial test for the
preference questions for the tested sample and Dermoplast ® spray
in the clinical study.

| | % that prefer the | % that prefer | |
|---|---|---|---|
| Question | tested sample | Dermoplast ® spray | p value |
| Q1a. Scent | 71.0% | 29.0% | 0.0196 |
| Q1b. Ease of use | 51.6% | 48.4% | 0.8575 |
| Q1c. The application | 45.2% | 54.8% | 0.5900 |
| (spray/nozzle) | | | |
| Q1d. The feel of the | 58.1% | 41.9% | 0.3692 |
| product on my skin | | | |
| Q1e. The numbing | 54.8% | 45.2% | 0.5900 |
| quality of the product | | | |
| Q3. After using both | 54.8% | 45.2% | 0.5900 |
| products, which one | | | |
| was your favorite? | | | |

Table 6 provides the dermal evaluation results for the tested sample. Table 7 provides the gynecological evaluation results for the tested sample. Under the described conditions, the tested sample did not demonstrate a potential for eliciting dermal irritation in the upper region of the lower extremities area and the surrounding dermis of the gynecological region. Further, under the conditions of this study and in this test population, the tested sample did demonstrate a potential for skin compatibility.

Moreover, under the conditions of this study and under the direction of a gynecologist, the nurse observed no clinically meaningful changes in the test site area/external genitalia of the subjects from the baseline visit, the Week 2 visit, and the Week 5/final visit. Additionally, the subjects did not report, with any clinically significant frequency, any comments or adverse events that would be considered unusual for each product. Therefore, the tested sample has been found safe for its intended use.

TABLE 6
Dermal evaluation results of the tested
sample from the clinical study.

| | Score for baseline visit, Week 2 visit, and Week 5/final visit | | | | | |
|---|---|---|---|---|---|---|
| | (baseline score/Week 2 score/Week 5 score)** | | | | | |
| | Left Inner Thigh | | | Right Inner Thigh | | |
| Subject | Erythema | Edema | Dryness | Erythema | Edema | Dryness |
| Number* | (Er) | (Ed) | (Dr) | (Er) | (Ed) | (Dr) |
| 01 | 0/0/0 | 0/0/0 | 0/0/0 | 0/0/0 | 0/0/0 | 0/0/0 |
| 02 | 0/0/0 | 0/0/0 | 0/0/0 | 0/0/0 | 0/0/0 | 0/0/0 |
| 03 | 0/0/0 | 0/0/0 | 0/0/0 | 0/0/0 | 0/0/0 | 0/0/0 |
| 04 | 0/0/0 | 0/0/0 | 0/0/0 | 0/0/0 | 0/0/0 | 0/0/0 |
| 06 | 0/0/0 | 0/0/0 | 0/0/0 | 0/0/0 | 0/0/0 | 0/0/0 |
| 07 | 0/0/0 | 0/0/0 | 0/0/0 | 0/0/0 | 0/0/0 | 0/0/0 |
| 08 | 0/0/0 | 0/0/0 | 0/0/0 | 0/0/0 | 0/0/0 | 0/0/0 |
| 09 | 0/0/0 | 0/0/0 | 0/0/0 | 0/0/0 | 0/0/0 | 0/0/0 |
| 10 | 0/0/0 | 0/0/0 | 0/0/0 | 0/0/0 | 0/0/0 | 0/0/0 |
| 11 | 0/0/0 | 0/0/0 | 0/0/0 | 0/0/0 | 0/0/0 | 0/0/0 |
| 12 | 0/0/0 | 0/0/0 | 0/0/0 | 0/0/0 | 0/0/0 | 0/0/0 |
| 13 | 0/0/0 | 0/0/0 | 0/0/0 | 0/0/0 | 0/0/0 | 0/0/0 |
| 14 | 0/0/0 | 0/0/0 | 0/0/0 | 0/0/0 | 0/0/0 | 0/0/0 |
| 15 | 0/0/0 | 0/0/0 | 0/0/0 | 0/0/0 | 0/0/0 | 0/0/0 |
| 16 | 0/0/0 | 0/0/0 | 0/0/0 | 0/0/0 | 0/0/0 | 0/0/0 |
| 17 | 0/0/0 | 0/0/0 | 0/0/0 | 0/0/0 | 0/0/0 | 0/0/0 |
| 18 | 0/0/0 | 0/0/0 | 0/0/0 | 0/0/0 | 0/0/0 | 0/0/0 |
| 19 | 0/0/0 | 0/0/0 | 0/0/0 | 0/0/0 | 0/0/0 | 0/0/0 |
| 20 | 0/0/0 | 0/0/0 | 0/0/0 | 0/0/0 | 0/0/0 | 0/0/0 |
| 21 | 0/0/0 | 0/0/0 | 0/0/0 | 0/0/0 | 0/0/0 | 0/0/0 |
| 22 | 0/0/0 | 0/0/0 | 0/0/0 | 0/0/0 | 0/0/0 | 0/0/0 |
| 23 | 0/0/0 | 0/0/0 | 0/0/0 | 0/0/0 | 0/0/0 | 0/0/0 |
| 24 | 0/0/0 | 0/0/0 | 0/0/0 | 0/0/0 | 0/0/0 | 0/0/0 |
| 25 | 0/0/0 | 0/0/0 | 0/0/0 | 0/0/0 | 0/0/0 | 0/0/0 |
| 26 | 0/0/0 | 0/0/0 | 0/0/0 | 0/0/0 | 0/0/0 | 0/0/0 |
| 27 | 0/0/0 | 0/0/0 | 0/0/0 | 0/0/0 | 0/0/0 | 0/0/0 |
| 28 | 0/0/0 | 0/0/0 | 0/0/0 | 0/0/0 | 0/0/0 | 0/0/0 |
| 29 | 0/0/0 | 0/0/0 | 0/0/0 | 0/0/0 | 0/0/0 | 0/0/0 |
| 30 | 0/0/0 | 0/0/0 | 0/0/0 | 0/0/0 | 0/0/0 | 0/0/0 |
| 31 | 0/0/0 | 0/0/0 | 0/0/0 | 0/0/0 | 0/0/0 | 0/0/0 |
| 32 | 0/0/0 | 0/0/0 | 0/0/0 | 0/0/0 | 0/0/0 | 0/0/0 |

*Subjects number 05 and 33 dropped from the study,
**Score scale: 0 = None; ± = Barely Perceptible; 1 = Slight; 2 = Mild; 3 = Moderate; 4 = Severe.

TABLE 7
Gynecological evaluation results of the tested sample from the clinical study.

| | Score for baseline visit, Week 2 visit, and Week 5/final visit | | | | | | | | |
|---|---|---|---|---|---|---|---|---|---|
| | (baseline score/Week 2 score/Week 5 score)** | | | | | | | | |
| | Labia Minora | | | Labia Majora | | | Vestibule/Perineum | | |
| Subject | Erythema | Edema | Dryness | Erythema | Edema | Dryness | Erythema | Edema | Dryness |
| number* | (Er) | (Ed) | (Dr) | (Er) | (Ed) | (Dr) | (Er) | (Ed) | (Dr) |
| 01 | 0/0/0 | 0/0/0 | 0/0/0 | 0/0/0 | 0/0/0 | 0/0/0 | 0/0/0 | 0/0/0 | 0/0/0 |
| 02 | 0/0/0 | 0/0/0 | 0/0/0 | 0/0/0 | 0/0/0 | 0/0/0 | 0/0/0 | 0/0/0 | 0/0/0 |
| 03 | 0/0/0 | 0/0/0 | 0/0/0 | 0/0/0 | 0/0/0 | 0/0/0 | 0/0/0 | 0/0/0 | 0/0/0 |
| 04 | 0/0/0 | 0/0/0 | 0/0/0 | 0/0/0 | 0/0/0 | 0/0/0 | 0/0/0 | 0/0/0 | 0/0/0 |
| 06 | 0/0/0 | 0/0/0 | 0/0/0 | 0/0/0 | 0/0/0 | 0/0/0 | 0/0/0 | 0/0/0 | 0/0/0 |
| 07 | 0/0/0 | 0/0/0 | 0/0/0 | 0/0/0 | 0/0/0 | 0/0/0 | 0/0/0 | 0/0/0 | 0/0/0 |
| 08 | 0/0/0 | 0/0/0 | 0/0/0 | 0/0/0 | 0/0/0 | 0/0/0 | 0/0/0 | 0/0/0 | 0/0/0 |
| 09 | 0/0/0 | 0/0/0 | 0/0/0 | 0/0/0 | 0/0/0 | 0/0/0 | 0/0/0 | 0/0/0 | 0/0/0 |
| 10 | 0/0/0 | 0/0/0 | 0/0/0 | 0/0/0 | 0/0/0 | 0/0/0 | 0/0/0 | 0/0/0 | 0/0/0 |
| 11 | 0/0/0 | 0/0/0 | 0/0/0 | 0/0/0 | 0/0/0 | 0/0/0 | 0/0/0 | 0/0/0 | 0/0/0 |
| 12 | 0/0/0 | 0/0/0 | 0/0/0 | 0/0/0 | 0/0/0 | 0/0/0 | 0/0/0 | 0/0/0 | 0/0/0 |
| 13 | 0/0/0 | 0/0/0 | 0/0/0 | 0/0/0 | 0/0/0 | 0/0/0 | 0/0/0 | 0/0/0 | 0/0/0 |
| 14 | 0/0/0 | 0/0/0 | 0/0/0 | 0/0/0 | 0/0/0 | 0/0/0 | 0/0/0 | 0/0/0 | 0/0/0 |
| 15 | 0/0/0 | 0/0/0 | 0/0/0 | 0/0/0 | 0/0/0 | 0/0/0 | 0/0/0 | 0/0/0 | 0/0/0 |
| 16 | 0/0/0 | 0/0/0 | 0/0/0 | 0/0/0 | 0/0/0 | 0/0/0 | 0/0/0 | 0/0/0 | 0/0/0 |
| 17 | 0/0/0 | 0/0/0 | 0/0/0 | 0/0/0 | 0/0/0 | 0/0/0 | 0/0/0 | 0/0/0 | 0/0/0 |
| 18 | 0/0/0 | 0/0/0 | 0/0/0 | 0/0/0 | 0/0/0 | 0/0/0 | 0/0/0 | 0/0/0 | 0/0/0 |
| 19 | 0/0/0 | 0/0/0 | 0/0/0 | 0/0/0 | 0/0/0 | 0/0/0 | 0/0/0 | 0/0/0 | 0/0/0 |
| 20 | 0/0/0 | 0/0/0 | 0/0/0 | 0/0/0 | 0/0/0 | 0/0/0 | 0/0/0 | 0/0/0 | 0/0/0 |
| 21 | 0/0/0 | 0/0/0 | 0/0/0 | 0/0/0 | 0/0/0 | 0/0/0 | 0/0/0 | 0/0/0 | 0/0/0 |
| 22 | 0/0/0 | 0/0/0 | 0/0/0 | 0/0/0 | 0/0/0 | 0/0/0 | 0/0/0 | 0/0/0 | 0/0/0 |
| 23 | 0/0/0 | 0/0/0 | 0/0/0 | 0/0/0 | 0/0/0 | 0/0/0 | 0/0/0 | 0/0/0 | 0/0/0 |
| 24 | 0/0/0 | 0/0/0 | 0/0/0 | 0/0/0 | 0/0/0 | 0/0/0 | 0/0/0 | 0/0/0 | 0/0/0 |
| 25 | 0/0/0 | 0/0/0 | 0/0/0 | 0/0/0 | 0/0/0 | 0/0/0 | 0/0/0 | 0/0/0 | 0/0/0 |
| 26 | 0/0/0 | 0/0/0 | 0/0/0 | 0/0/0 | 0/0/0 | 0/0/0 | 0/0/0 | 0/0/0 | 0/0/0 |
| 27 | 0/0/0 | 0/0/0 | 0/0/0 | 0/0/0 | 0/0/0 | 0/0/0 | 0/0/0 | 0/0/0 | 0/0/0 |
| 28 | 0/0/0 | 0/0/0 | 0/0/0 | 0/0/0 | 0/0/0 | 0/0/0 | 0/0/0 | 0/0/0 | 0/0/0 |
| 29 | 0/0/0 | 0/0/0 | 0/0/0 | 0/0/0 | 0/0/0 | 0/0/0 | 0/0/0 | 0/0/0 | 0/0/0 |
| 30 | 0/0/0 | 0/0/0 | 0/0/0 | 0/0/0 | 0/0/0 | 0/0/0 | 0/0/0 | 0/0/0 | 0/0/0 |
| 31 | 0/0/0 | 0/0/0 | 0/0/0 | 0/0/0 | 0/0/0 | 0/0/0 | 0/0/0 | 0/0/0 | 0/0/0 |
| 32 | 0/0/0 | 0/0/0 | 0/0/0 | 0/0/0 | 0/0/0 | 0/0/0 | 0/0/0 | 0/0/0 | 0/0/0 |

*Subjects number 05 and 33 dropped from the study.
**Score scale: 0 = None; 1 = Minimal; 2 = Moderate; 3 = Severe/Marked.

Although the foregoing description is believed to be accurate, it is provided for exemplary purposes and is not intended to limit the scope of the invention.

1. A solution for application to a perineal area of a woman after giving birth, the solution comprising:
a topical anesthetic; and
at least one herbal ingredient.

2. The solution of claim 1, wherein the topical anesthetic comprises lidocaine.

3. The solution of claim 1, wherein the topical anesthetic is selected from the group consisting of Articaine, Benzocaine, Bupivacaine, Chloroprocaine, Cocaine, Dibucaine, Dyclonine, Etidocaine, Lignocaine, Mepivacaine, Prilocaine, Procaine, Tetracaine, Xylocaine, and any combinations thereof.

4. The solution of claim 1, wherein the topical anesthetic has a concentration from about 0.05% to about 5% of the solution.

5. The solution of claim 1, wherein the at least one herbal ingredient comprises multiple herbal ingredients, and wherein each of the multiple herbal ingredients has a concentration of about 5% or less of the solution.

6. The solution of claim 1, wherein the at least one herbal ingredient is selected from the group consisting of Hamamelis virginiana (witch hazel) Extract, Aloe Barbadensis Leaf juice, Lavender Oil, Calendula officinalis Flower Extract, Chamomile Recutita (Matricaria) Flower Extract, Tea Tree leaf oil, Mentha Piperita (peppermint) oil, Prunus amygdalus dulcis (sweet almond) oil, and any combinations thereof.

7. The solution of claim 1, further comprising one or more stabilizing ingredients selected from the group consisting of Caprylyl/Capryl Wheat Bran/Straw Glycosides, Fusel Wheat Bran/Straw Glycosides, Cellulose gum, Poloxamer1, PEG 40 Hydrogenated Castor Oil, Polyglyceryl-5 Oleate, Sodium Cocoyl Glutamate, Glyceryl Caprylate, Sodium phytate, EDTA, Sodium benzoate, Potassium Sorbate, Potassium benzoate, Poloxamer188, Sorbic acid, Calcium sorbate, calcium propionate, and any combinations thereof.

8. The solution of claim 1, wherein the solution is clear.

9. The solution of claim 1, further comprising one or more active antimicrobial agents selected from the group consisting of iodine, Chlorhexidine, honey, PHMB, silver compounds (e.g., silver nitrate, silver sulphadiazine, or silver acetate), bacitracin, polymyxin, hyaluronic acid, octenidine, and any combination thereof.

10. The solution of claim 1, further comprising one or more stabilizing ingredients selected from the group consisting of Caprylyl/Capryl Wheat Bran/Straw Glycosides, Fusel Wheat Bran/Straw Glycosides, Cellulose gum, Poloxamer1, PEG 40 Hydrogenated Castor oil, Polyglyceryl-5 Oleate, Sodium Cocoyl Glutamate, Glyceryl Caprylate, Sodium phytate, EDTA, Sodium benzoate, Potassium Sorbate, Potassium benzoate, Poloxamer188, Sorbic acid, Calcium sorbate, calcium propionate, and any combinations thereof.

11. The solution of claim 1, wherein a viscosity of the solution is from about 0.0095 to about 3.0000 poise.

12. The solution of claim 1, wherein the solution is opaque and substantially free of precipitates.

13. A method for treating a perineal area of a woman after giving birth, the method comprising:
spraying a solution from a pump sprayer onto the perineal area, the solution comprising:
a topical anesthetic; and
at least one herbal ingredient.

14. The method of claim 13, wherein the solution adheres to a skin of the perineal area.

15. The method of claim 13, wherein the solution is clear.

16. The method of claim 13, wherein the solution is opaque.

17. The method of claim 13, wherein the at least one herbal ingredient is selected from the group consisting of Hamamelis virginiana (witch hazel) Extract, Aloe Barbadensis leaf juice, Lavender oil, Calendula officinalis flower extract, Chamomile Recutita (Matricaria) flower extract, Tea tree leaf oil, Mentha Piperita (peppermint) oil, Prunus amygdalus dulcis (sweet almond) oil, and any combinations thereof.

18. The method of claim 13, wherein the solution further comprises one or more active antimicrobial agents.

19. The method of claim 13, wherein the solution is sprayed as a mist.

20. The method of claim 13, wherein the topical anesthetic has a concentration from about 0.05% to about 5% of the solution.
